(19) United States
(12) Patent Application Publication
(10) Pub. No.: US 2024/0141275 A1
(43) Pub. Date: May 2, 2024

(54) SYSTEMS AND METHODS FOR EXPANSION AND DIFFERENTIATION OF THERAPEUTIC CELLS IN BIOREACTORS

(71) Applicant: PBS Biotech, Inc., Camarillo, CA (US)

(72) Inventor: Chanyong Brian LEE, Thousand Oaks, CA (US)

(21) Appl. No.: 16/517,902

(22) Filed: Jul. 22, 2019

Related U.S. Application Data
(63) Continuation-in-part of application No. 16/282,129, filed on Feb. 21, 2019, now abandoned.
(60) Provisional application No. 62/634,077, filed on Feb. 22, 2018.

(51) Int. Cl. C12M 1/00

(52) U.S. Cl. CPC C12M 29/18; C12M 29/14; C12M 29/26

(57) ABSTRACT

Systems and methods for complete medium exchange using two bioreactors and at least one external separation and retention device designed for therapeutic cells grown as aggregates, on the surface of microcarriers, or as single cells, for scalable expansion and/or directed differentiation.

### RELATED APPLICATION INFORMATION

This application is a continuation-in-part of U.S. patent application Ser. No. 16/282,129, filed Feb. 21, 2019, which claims priority from U.S. Provisional Patent Application No. 62/634,077, filed Feb. 22, 2018, titled BIOREACTOR SYSTEMS AND METHODS FOR DIFFERENTIATION OF CELLS, which are expressly incorporated herein by reference.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by anyone of the patent disclosure as it appears in the Patent and Trademark Office patent files or records, but otherwise reserves all copyright and trade dress rights whatsoever.

### FIELD OF THE INVENTION

Systems and methods for complete and efficient medium exchange, particularly at large volumetric scales, for expansion and directed differentiation of therapeutic cells, including cell aggregates, microcarriers with attached cells, or single cells, in bioreactors.

### BACKGROUND

Bioprocesses involving cells grown in suspension culture while being mixed inside bioreactors are being developed for a wide range of cell and gene therapy applications. Depending on their type and properties, these therapeutic cells proliferate while clumped together as aggregates, attached to the surface of microcarriers (MCs), or suspended as individual cells. As part of the upstream process, the liquid medium that cells are suspended in will need to be exchanged, i.e., spent medium removed and fresh medium added. This is necessary to replenish nutrients or to supply specific growth hormones, as well as to eliminate metabolic waste and other unwanted byproducts. Upstream processes that require medium exchanges are cell expansion (increasing the total number of cells) and directed differentiation (directing pluripotent cells to turn into a specific cell type).

There are various techniques for performing medium exchange in bioreactors. One common method is to pause agitation and allow all the cell aggregates, MCs with cells growing on their surfaces, or suspended single cells to settle by gravity to the bottom of the bioreactor. Once a bed of settled cell aggregates, MCs with attached cells, or single cells is formed, the supernatant of spent medium is removed, fresh medium is added, and agitation is restarted to resuspend the cell aggregates, MCs with attached cells, or single cells. There are two potential issues with this method, which become exacerbated as bioreactor working volume increases.

First, the temporary cessation of mixing can lead to cell damage through unwanted agglomeration, nutrient starvation, and deviation of key process parameters such as temperature, pH, and dissolved oxygen levels. Second, it is difficult to completely remove all the spent medium, as withdrawing supernatant too close to the bed of settled cell aggregates, MCs with attached cells, or single cells can result in cell loss, while using a filtered retention device can lead to clumping, clogging, and cell damage. Certain processes, such as multi-step directed differentiation of pluripotent stem cell (PSC) aggregates, can have reduced efficiency and yields if previously used growth factors remaining in residual medium are not completely removed between each differentiation step. A process that can achieve complete medium exchange in large scale bioreactors while minimizing potential damage to cells would greatly improve the yield and efficiency of processes for cell expansion and differentiation and thus be an invaluable tool for commercial manufacturing of emerging cell and gene therapies.

PSCs can be derived from human embryos or by inducing pluripotency in adult somatic cells. The distinguishing characteristic of PSCs is their ability to differentiate into virtually any cell type in the human body, which makes them a promising cell therapy tool to potentially treat a wide variety of different disease indications. Furthermore, PSCs can grow indefinitely as cell aggregates in culture, which is critical to meet dosage needs that can range from millions to even billions of cells per person. Attempting to produce a huge magnitude of cells at commercial scale using traditional 2D manufacturing platforms would be extremely cost prohibitive and thus infeasible. Instead, 3D suspension culture in a bioreactor represents the best option for development and scale-up of PSC bioprocesses.

One requirement of PSC manufacturing is directed differentiation steps performed in vitro which will guide the cells to turn into a target cell type. First, a cell expansion phase occurs in a bioreactor. The differentiation phase entails multiple medium exchange steps in situ in the bioreactor where the expansion phase was completed. While complete medium exchange can be achieved relatively easily and completely at small scale in R&D settings, accomplishing it at large scale for commercial manufacturing presents a major challenge.

In order to successfully achieve commercial manufacturing of cell therapy products, there is a need for a rapid, efficient, and scalable medium exchange technique for bioprocesses involving therapeutic cells.

### SUMMARY OF THE INVENTION

The present application discloses methodology for complete medium exchange, with minimal damage, to therapeutic cells that grow as cell aggregates, on the surface of microcarriers, or as suspended single cells, using at least two bioreactors and one external device designed for separation and retention of therapeutic cells. Instead of the therapeutic cells remaining inside a bioreactor during medium exchange, they are instead removed, in spent medium, to an external separation and retention device that will concentrate and thoroughly wash them with fresh medium before returning them to a different bioreactor that has been prepared with the identical medium used for washing.

Various types of separation and retention devices can be used for this process. Methods of their operation can include centrifugation (conventional or continuous anti-centrifugal force), acoustic precipitation, or simple filtering. The primary purpose of these devices is to isolate, concentrate, and wash therapeutic cells such as cell aggregates, MCs with attached cells, or suspended single cells.

This novel method for complete medium exchange eliminates the issues associated with gravity-based settling and enables large-scale manufacturing of therapeutic cells. The technique is particularly useful for directed differentiation of pluripotent stem cells (PSCs) which grow as cell aggregates.

The envisioned embodiment utilizes at least two bioreactors and one external device designed for separation and retention of therapeutic cells. After an expansion or differentiation process step is completed in a first bioreactor, spent medium containing the therapeutic cells is transferred to a separation and retention device where the therapeutic cells are collected and concentrated. They are then washed with a new medium required for the next process step and then transferred immediately to a second bioreactor, which has already been prefilled with identical medium and preconditioned for necessary parameters such as temperature, pH, and dissolved oxygen.

A prolonged lag phase during cell growth can be detrimental to overall expansion efficiency, thus it is desirable to have a preconditioned bioreactor ready for the therapeutic cells to immediately return to after they leave the separation and retention device. The total time to prefill and precondition a bioreactor will likely take longer than a concentration and washing step in the separation and retention device, especially at larger bioreactor volumes. Therefore, the prefilling and preconditioning of a second bioreactor can begin while an expansion step is ongoing in the first bioreactor. After all therapeutic cells leave the first bioreactor, it becomes available to be prefilled and preconditioned as the third bioreactor in sequence. The now “third” (previously first) bioreactor should be ready to receive the therapeutic cells after they move from the second bioreactor, into the separation and retention device, and back out of the device. Alternatively, if the first bioreactor cannot be prepared in time, a completely new, third bioreactor can be prefilled and preconditioned instead; any number of bioreactors can be used as dictated by the time required for prefilling and preconditioning a bioreactor. This time will only increase as the cell manufacturing process and bioreactor volumes scale up. By using the separation and retention device as a bridge to cycle between bioreactors, multiple complete medium exchange steps can be accomplished efficiently and quickly, even at large scales.

Another embodiment utilizes just one bioreactor and one external separation device. Depending on the time required for concentrating and washing therapeutic cells in the external device, a single bioreactor can be used for complete medium exchange. After all the spent medium is removed from the bioreactor, it must be refilled and conditioned before therapeutic cells are finished being concentrated and washed in the external device and ready to be returned. Otherwise the same problems associated with settling methods, where cells are idle without proper mixing culture conditions, can occur. However, as bioreactor volume increases so too does the time required to fill with media and precondition. Thus it may be more difficult to avoid negative impact to therapeutic cells using this method compared to a method with two or more bioreactors.

### DESCRIPTION OF THE DRAWINGS

FIG. 1 shows a multi-stage directed differentiation process for generation of insulin-producing pancreatic β cells in vitro;

FIG. 2 shows a system of the present application with two vertical-wheel bioreactors and one generic example of an external device designed for separation and retention of therapeutic cells such as cell aggregates, microcarriers (MCs) with cells attached to their surfaces, or suspended single cells;

FIG. 3 shows a system of the present application with multiple vertical-wheel bioreactors and separation and retention devices designed for separation and retention of therapeutic cells.

### DETAILED DESCRIPTION

The present application provides systems and methods for expansion and differentiation of cells grown as aggregates, on the surface of MCs, or as single suspended cells in bioreactors using complete medium exchanges. In a typical embodiment, the operation of two bioreactors is alternated, with an external separation and retention device acting as bridge between them. However, the concepts described herein may also be facilitated using more than two bioreactors, as well as multiple separation and retention devices.

Furthermore, although the medium exchange techniques described herein are particularly useful for large-scale differentiation of pluripotent stem cells (PSCs), they may also be utilized in the expansion of other anchorage-dependent cells, such as mesenchymal stem cells (MSCs) or human primary cells grown on microcarriers or as cell aggregates.

Additionally, various types of bioreactors may be used for expansion and differentiation of cells. In a preferred embodiment, a vertical-wheel bioreactor is used for its relatively low shear rate which minimizes potential damage to anchorage-dependent cells. However, other bioreactor configurations with alternative agitation methods, such as stirred-type impellers, may be used.

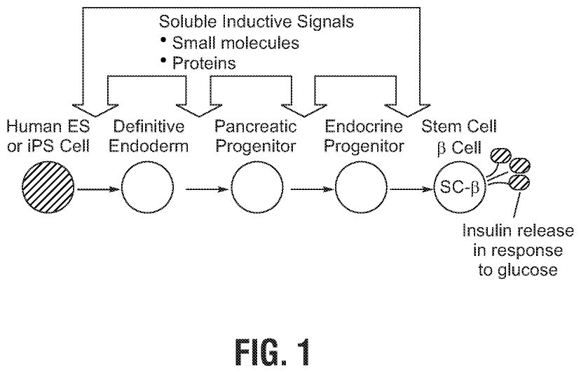

As mentioned, a unique requirement of PSC manufacturing is the directed differentiation steps which will guide the cells to turn into a target cell type. For instance, FIG. 1 shows a sequence of four steps where various growth factors are used to direct pluripotent cells through various stages until insulin-producing pancreatic β cells are produced.

Commonly, the differentiation phase entails multiple medium exchange steps in situ in the same bioreactor where the expansion phase was completed. Between each differentiation step, PSC aggregates settle to the bottom of the vessel, spent medium is removed, and new medium along with specific growth factors are added for the next differentiation step. There are two problems, as previously mentioned. First, settling of the cell aggregates at the bottom of the bioreactor can lead to cell damage. Second, it is difficult to completely withdraw spent medium, whether it is from supernatant above the level of settled cell aggregates or through a filtered cell retention device. Any carried-over medium from a previous differentiation step may reduce the efficiency of the subsequent differentiation step using a completely different medium. Ideally, the growth factors that are no longer desired and any residual byproducts need to be completely removed before the addition of a different medium with new growth factors for the next differentiation step.

To solve the problems inherent to large-scale differentiation of cell aggregates, directed differentiation requiring complete medium exchange can be performed in vertical-wheel bioreactor systems, in conjunction with an external separation and retention device. Instead of allowing cell aggregates to settle, the culture medium is removed to a device connected outside of a vertical-wheel bioreactor. Within the separation and retention device the cell aggregates can be concentrated and washed with buffer and new medium for the next differentiation step, which will completely remove spent medium, obsolete growth factors, and byproducts. The concentrated and washed cell aggregates are then returned immediately to a different vertical-wheel bioreactor which has been prefilled with the same differentiation medium used for washing and is ready for the next differentiation step. In this way, PSC aggregates can be quickly and efficiently differentiated at large volumes without damaging the cells. This new, large-scale differentiation process will improve the overall yield of target cell production as well as the manufacturability of differentiated cells at commercial scale.

Various separation and retention devices could be used for this process such as: centrifugation (conventional or continuous anti-centrifugal force), acoustic precipitation, or simple filtering. Any of these devices can be easily coupled with a vertical-wheel bioreactor to allow for complete medium exchanges, thus improving the yield and efficiency of cell expansion or differentiation even at large scales. An entire cell manufacturing process, including the cell expansion and directed differentiation phases, can be performed and optimized at large scales in single-use, vertical-wheel bioreactors.

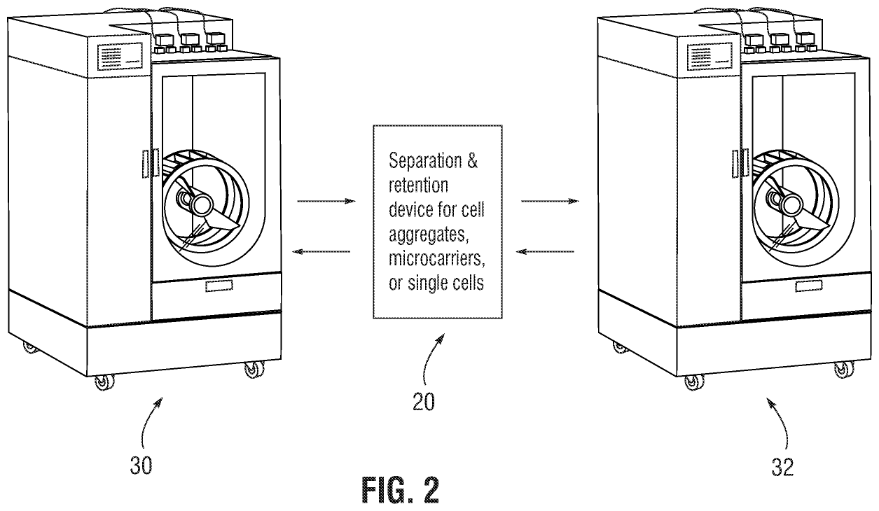

FIG. 2 shows a system of the present application with a generic example of a separation and retention device 20 and two bioreactors 30, 32. A first bioreactor 30, which in the illustrated embodiment is a vertical-wheel bioreactor, is used to grow, for example, PSC aggregates in an expansion medium. The low-shear environment of vertical-wheel bioreactors allows for these aggregates to reach high cell densities at large scale (e.g. 50L working volume) during the expansion phase.

When transitioning from the expansion to the differentiation phase, the PSC aggregates can be harvested from the first bioreactor 30 and transferred to the separation and retention device 20. Various types of separation and retention devices can be used for this process. Methods of their operation can include centrifugation (conventional or continuous anti-centrifugal force), acoustic precipitation, or simple filtering. The primary purpose of these devices is to isolate, concentrate, and wash cell aggregates, MCs with attached cells, or suspended single cells. One example of a separation and retention devices is the kSep scalable, single-use automated centrifugation system available from Sartorius Stedim Plastics GmbH of Goettingen, Germany.

As spent expansion medium flows into the separation and retention device 20, the PSC aggregates will ideally be concentrated in a small volume of spent medium without becoming compacted. Once the aggregates are concentrated, the fluid flow of spent medium will be replaced with the first differentiation medium. The cell aggregates are thus washed and the old expansion medium is completely removed and replaced with differentiation medium.

Finally, the PSC aggregates will be discharged from the separation and retention device 20 into the second bioreactor 32 that has already been prefilled and preconditioned with the first differentiation medium. Preconditioning means calibrating the bioreactor for key parameters, such as temperature, pH, and dissolved oxygen, necessary for the next process step. As preconditioning typically takes longer than the time required for concentrating and washing in the separation and retention device, the second bioreactor can begin its prefilling and preconditioning process before the PSC aggregates leave the first bioreactor. The prefilled and preconditioned second bioreactor 32 will therefore be ready to be inoculated with washed PSC aggregates immediately after they leave the separation and retention device 20.

Once a differentiation step is completed in the second bioreactor 32, the PSC aggregates (now comprised of a different cell type) can be concentrated and washed in the same manner using the separation and retention device 20 and transferred back to the first bioreactor 30 which has been prefilled and preconditioned with yet another differentiation medium. By alternating back and forth between two bioreactors via the separation and retention device 20, complete medium exchange can be continuously achieved throughout a multi-step differentiation process without negatively impacting cell viability, quality, pluripotency, or yield. This back-and-forth process may be performed as many times as required, such as to perform the sequence of FIG. 1.

If the time to precondition a bioreactor is shorter than the time needed for concentrating and washing step, a single bioreactor can be used for that particular expansion and/or differentiation step. Depending on preconditioning time requirements for different scale steps of the bioprocesse, any number of bioreactors and external devices can be used in different combinations as needed, particularly for large scale manufacturing.

One potential process variation could involve following the previously described differentiation process until cell aggregates of a particular intermediate cell type are produced. At this point, only a portion of the cell aggregates would be subjected to a particular growth factor, with the remaining portion subjected to a different growth factor. This would result in two differentiation pathways that end in different final target cell types.

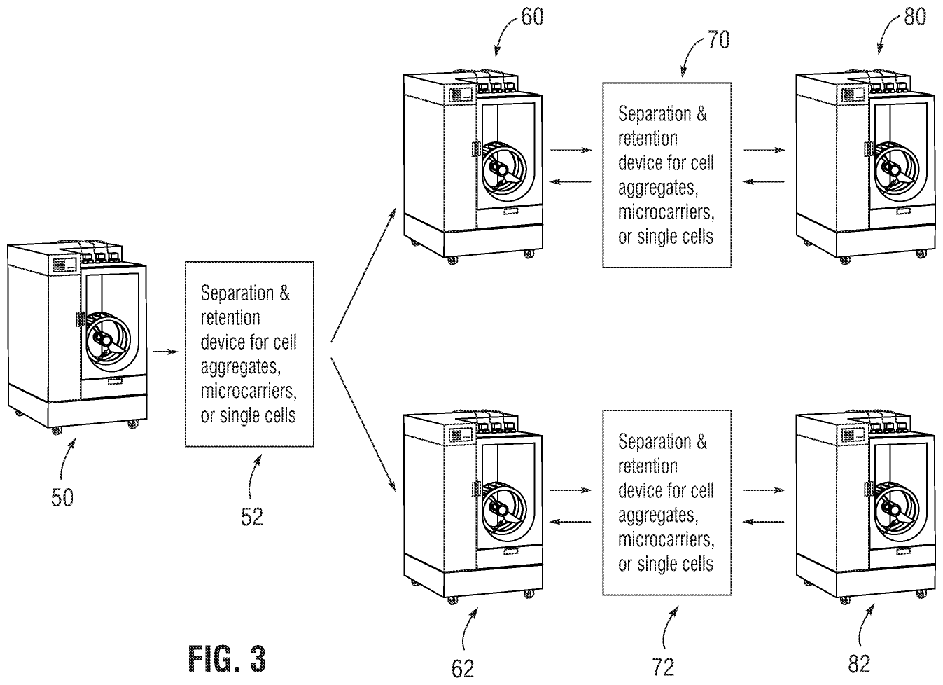

For example, FIG. 3 shows a system of the present application with multiple separation and retention devices and bioreactors. In this case, each parallel pathway would require at least two bioreactors and one separation and retention device for the alternating medium exchange process described with reference to FIG. 2.

More specifically, a first bioreactor 50 is used to start the process by growing a batch of PSC aggregates. The aggregates are transferred to a first generic example of a separation and retention device 52. The batch of aggregates may be washed with a first differentiation medium A, and then a portion of them will be transferred to a second bioreactor 60 which has been pre-filled with medium A. The remaining aggregates in the separation and retention device 52 will then be washed with a second differentiation medium B and transferred to a third bioreactor 62 which been pre-filled with medium B. The second bioreactor 60 and third bioreactor 62 are then operated in parallel to perform their respective differentiation steps.

Subsequently, the PSC aggregates in the second and third bioreactors 60, 62 will be transferred to separation and retention devices 70, 72, respectively. It is possible for the first device 52 to function as either device 70, 72, or both. That is, a single separation and retention device could be used in multiple differentiation pathways if the timing of concentration and washing steps are staggered. However, to maintain clarity, the separation and retention devices 70, 72 in the two parallel pathways are identified uniquely in FIG. 3.

PSC aggregates are transferred from the second bioreactor 60 to the separation and retention device 70 for concentrating and washing and are then transferred to a fourth bioreactor 80 for differentiation. PSC aggregates are transferred from the third bioreactor 62 to the separation and retention device 72 and are then transferred to a fifth bioreactor 82. Either the fourth or fifth bioreactor 80, 82 may be the first bioreactor 50 which was used to start the process, if bioreactor 50 was sufficiently prefilled and preconditioned ahead of time. Likewise, instead of transferring aggregates to the fourth and fifth bioreactors 80, 82, the output from the devices 70, 72 may be transferred back to the second and third bioreactors 60, 62. The reader will appreciate the various permutations available, limited only by the time needed to prefill and precondition each bioreactor or by the number of available bioreactors. Multiple differentiation pathways branching off from multiple intermediate precursor cell types is also possible, with each pathway requiring its own unique growth factors, bioreactors, and separation and retention devices, and ending in different final target cells.

One factor to consider is the method of medium transfer between bioreactor and separation and retention device. Peristaltic pumps are commonly used to move liquid medium through flexible plastic tubing. However, the physical squeezing action of the pump may crush cell aggregates with diameters greater than 400 micrometers or cause MCs to grind against each other, damaging surface-bound cells. Viability of suspended single cells is unlikely to be significantly affected due to the individual cells being smaller than both the eddy sizes of fluid turbulence and the gap between the interior sides of squeezed tubing.

In lieu of using pumps, medium transfer can be achieved by creating a difference in internal pressure between the bioreactor and separation and retention device. Liquid transfer by differential pressure can be achieved relatively easily when the vessels are constructed with materials, such as stainless steel, to withstand internal pressure. Single-use bioreactors that have rigid frames to encapsulate their flexible plastic film vessels can also be successfully pressurized without risk of operational hazards. In preparation for medium transfer, the harvest valve of a bioreactor is connected aseptically to the separation and retention device through a clamped plastic tubing. Gas is then pumped into the headspace of the bioreactor to pressurize it. When the clamp is opened, liquid containing cell aggregates or MCs with attached cells will flow to the lower pressure external device. The transfer rate can be controlled by adjusting the gas pressure applied in the bioreactor. A second tubing that connects the separation and retention device to a second bioreactor will remain clamped during this first pressurized transfer step.

Another method for medium transfer involves a system of lifts that can be used to change the heights of bioreactors relative to each other. Thus if one bioreactor is raised high enough compared to the other, gravity will cause therapeutic cells to fall down through tubing from the raised bioreactor into the lower one.

Once the cell aggregates or MCs with attached cells are washed, the separation and retention device can be pressurized, and then the second clamp will be opened to allow flow out of the device. By utilizing this technique, aggregates and MCs can avoid the potentially damaging effects of physical pumping.

As used herein, “plurality” means two or more. As used herein, a “set” of items may include one or more of such items. As used herein, whether in the written description or the claims, the terms “comprising”, “including”, “carrying”, “having”, “containing”, “involving”, and the like are to be understood to be open-ended, i.e., to mean including but not limited to. Only the transitional phrases “consisting of” and “consisting essentially of”, respectively, are closed or semi-closed transitional phrases with respect to claims. Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. As used herein, “and/or” means that the listed items are alternatives, but the alternatives also include any combination of the listed items.

1. A method of cell expansion of therapeutic cells grown as cell aggregates, attached to the surfaces of microcarriers, or as suspended single cells, comprising:
a. suspending therapeutic cells including cell aggregates, microcarriers with attached cells, or single cells in a first medium inside a first bioreactor having a first working volume;
b. expanding or differentiating the therapeutic cells in the first bioreactor;
c. prefilling a second bioreactor having a second working volume with a second medium;
d. preconditioning process parameters for the second bioreactor containing the second medium;
e. transferring all of the first medium containing the therapeutic cells from the first bioreactor to an external device for cell concentration and medium exchange;
f. concentrating and washing the therapeutic cells in the external device using the second medium;
g. transferring the concentrated and washed therapeutic cells from the external device to the prefilled and preconditioned second bioreactor;
h. expanding or differentiating the transferred concentrated and washed therapeutic cells in the second bioreactor until a desired expansion of the therapeutic cells is complete.

2. The method of claim 1, further comprising:
i. prefilling a third bioreactor having a third working volume with a third medium;
j. preconditioning process parameters for the third bioreactor containing the third medium;
k. transferring all of the second medium containing the therapeutic cells from the second bioreactor to the external device;
l. concentrating and washing the therapeutic cells in the external device using the third medium;
m. transferring the concentrated and washed therapeutic cells from the external device to the prefilled and preconditioned third bioreactor; and
n. expanding or differentiating the concentrated and washed therapeutic cells in the third bioreactor until a desired expansion of the therapeutic cells is complete.

3. The method of claim 2, wherein the third bioreactor is the first bioreactor and the method further comprises cleaning, prefilling and preconditioning the first bioreactor prior to step i.

4. The method of claim 1, further including repeating steps c. through h. one or more times using one or more subsequent bioreactors and subsequent mediums in place of the second bioreactor and second medium.

5. The method of claim 2, wherein all of the first, second, and third working volumes are identical.

6. The method of claim 2, wherein the second working volume is larger than the first working volume and the third working volume is larger than the second working volume.

7. The method of claim 4, wherein step e. is performed using only a single external device.

8. The method of claim 4, wherein each time step e. is performed a different external device is used.

9. The method of claim 1, wherein the steps of transferring are done using pumps or a fluid pressure differential.

10. A method of directed differentiation of therapeutic cells growing as cell aggregates, on the surfaces of microcarriers, or as suspended single cells, comprising:
a. suspending therapeutic cells including cell aggregates, microcarriers with attached cells, or single cells in a first medium in a first differentiation medium that contains first growth factors inside a first bioreactor having a first working volume;
b. prefilling a second bioreactor with a second differentiation medium and adding second growth factors that will be used in a subsequent differentiation step;
c. preconditioning process parameters for the second bioreactor;
d. transferring all of the first differentiation medium, growth factors, and therapeutic cells from the first bioreactor to an external device for cell concentration and medium exchange;
e. concentrating and washing the therapeutic cells using the second differentiation medium, to remove previous growth factors, in the external device; and
f. transferring the concentrated and washed therapeutic cells from the external device to the preconditioned second bioreactor.

11. The method of claim 10, further comprising:
g. prefilling a third bioreactor having a third working volume with a third medium;
h. preconditioning process parameters for the third bioreactor containing the third medium;
i. transferring all of the second medium containing the therapeutic cells from the second bioreactor to the external device;
j. concentrating and washing the therapeutic cells in the external device using the third medium; and
k. transferring the concentrated and washed therapeutic cells from the external device to the prefilled and preconditioned third bioreactor.

12. The method of claim 11, wherein the third bioreactor is the first bioreactor and the method further comprises cleaning, prefilling and preconditioning the first bioreactor prior to step g.

13. The method of claim 10, further including repeating steps b. through f. one or more times using one or more subsequent bioreactors and subsequent fresh mediums in place of the second bioreactor and second medium.

14. The method of claim 13, wherein all of the bioreactors used have identical working volumes.

15. The method of claim 13, wherein step e. is performed using only a single external device.

16. The method of claim 13, wherein each time step d. is performed a different external device is used.

17. The method of claim 10, wherein the steps of transferring are done using pumps or a fluid pressure differential.

18. The method of claim 1, wherein step e. transferring all of the first medium containing the therapeutic cells from the first bioreactor to an external device, comprises transferring all of the first medium containing the therapeutic cells from the first bioreactor to multiple external devices.

19. The method of claim 18, wherein the multiple external devices are disposed in parallel.

20. The method of claim 10, wherein step d. transferring all of the first differentiation medium, growth factors, and therapeutic cells from the first bioreactor to an external device, comprises transferring all of the first differentiation medium, growth factors, and therapeutic cells from the first bioreactor to multiple external devices.

21. The method of claim 20, wherein the multiple external devices are disposed in parallel.

22. The method of claim 1, wherein step c. prefilling the second bioreactor having the second working volume with the second medium and step d. preconditioning the process parameters for the second bioreactor containing the second medium, begin before step e. transferring all of the first medium containing the therapeutic cells from the first bioreactor to an external device.

23. The method of claim 10, wherein step b. prefilling the second bioreactor with the second differentiation medium and adding second growth factors that will be used in the subsequent differentiation step, and step c. preconditioning the process parameters for the second bioreactor, begin before step d. transferring all of the first differentiation medium, growth factors, and therapeutic cells from the first bioreactor to an external device.
